(19) United States
(12) Patent Application Publication
(10) Pub. No.: US 2012/0183587 A1
(43) Pub. Date: Jul. 19, 2012

(54) FLAVONOL COMPOSITIONS

(76) Inventor: Mitsunori Ono, Lexington, MA (US)

(21) Appl. No.: 13/350,266

(22) Filed: Jan. 13, 2012

Related U.S. Application Data
(60) Provisional application No. 61/433,777, filed on Jan. 18, 2011.

(51) Int. Cl. A61K 31/353; A61K 9/10; A61K 8/04; A61K 8/49

(52) U.S. Cl. 424/401; 514/456; 424/400

(57) ABSTRACT

Described are 3-hydroxyflavonol compositions with improved water-solubility and methods of making the compositions.

### CROSS-REFERENCE TO RELATED APPLICATION

This application claims priority to U.S. Provisional Application No. 61/433,777, filed on Jan. 18, 2011, the content of which is incorporated herein by reference in its entirety.

### BACKGROUND

The diversity of flavonols, flavonoids that have the 3-hydroxyflavone backbone (i.e., 3-hydroxy-2-phenylchromen-4-one), stems from different positions of the phenolic-OH groups. Flavonols are distinct from flavanols (e.g., catechin), which belong to another class of flavonoids. They are generally isolated from the rinds of oranges, tangerines, lemons, limes, kumquats and grapefruits by commercial extraction methods. In Western populations, daily intake of flavonols is estimated to be in the range of 20-50 mg. Individual intake varies depending on the diet.

Flavonols are involved in a number of biological processes. For example, they play a role in homeostasis of the walls of small blood vessels and maintenance of normal blood vessel conditions by decreasing capillary permeability and fragility. Flavonols also act as a histamine release blocker, a xanthine oxidase inhibitor, an aldose reductase inhibitor, a phospholiphase A2 and lipoxygenase inhibitor, an aerobic glycosis inhibitor, a singlet oxygen quencher, and a tumor necrosis factor potentiator.

Despite the promise for a variety of medicinal, dietary, and cosmetic applications, the utility of flavonols has been limited by their poor absorption into the bloodstream due to, at least in part, their low water-solubility or water-affinity under physiological conditions. For example, one of the representative natural flavonols, quercetin (i.e., 3,3′,4′,5,7-O penta-hydroxyflavone), is absorbed to the extent of only about 1% from an oral dose. See, e.g., Guglen et al., Eur. J. Clin. Pharmacol., 9, 229-234 (1975). There is a need for flavonols in forms that offer higher water solubility and thus higher bioavailability.

### SUMMARY

This present invention is based on the unexpected discovery that a powder composition including an alkali metal salt of a 3-hydroxyflavone has improved water solubility and bioavailability. Accordingly, within the scope of this invention are compositions including an alkali metal salt of a 3-hydroxyflavone and methods of making the compositions.

In one aspect, this invention relates to an edible powder composition including an alkali metal salt of a 3-hydroxyflavone, an alkali metal salt of an organic acid, a water-soluble antioxidant, and optionally, a water-soluble anti-deliquescent agent. This powder composition, when dissolved in water at 0.2%/w/v, can result in a solution that has a pH of 8.5 to 11.5, e.g., 9.0 to 11.0. The alkali metal can be sodium or potassium.

Examples of the 3-hydroxyflavone include quercetin, azaleatin, fisetin, galangin, gossypetin, kaempferide, kaempferol, isorhamnetin, mortin, myricetin, natsudaidain, pachypodol, rhamnazin, and rhamnetin. Two or more alkaline metal salts of different 3-hydroxyflavones can be included in the powder composition.

The water-soluble anti-deliquescent agent included in the composition can be a gelatin or a polysaccharide, e.g., acid treated porcine-derived gelatin, maltodextrin, or cluster dextrin. The organic acid is preferably a weak organic acid, e.g., citric acid and acetic acid. The antioxidant can be, e.g., vitamin C.

The amounts of the various components of the powder composition can vary, depending on, for example, the specific 3-hydroxyflavone and the desired properties (e.g., stability) of the powder composition.

Generally, the composition can contain 10% to 40% (e.g., 15% to 35%) of the alkali metal salt of a 3-hydroxyflavone, 2% to 20% (e.g., 5% to 20%) of the anti-deliquescent agent, and 10% to 40% (e.g., 15% to 35%) of the water-soluble antioxidant.

In another aspect, the present invention relates to a suspension composition containing (1) a solvent and (2) the powder composition described above suspended in the solvent, the suspension composition having a pH of 2.5 to 8.5 (e.g., 3.0 to 7.5). The suspension composition can contain 0.01% to 5% of the powder composition. The solvent can be, for example, water or an aqueous alcohol containing up to 20% alcohol.

The suspension composition can also include a surfactant (e.g., nonionic sugar emulsifier), or a water-soluble polymer (e.g., a polyglycerine fatty acid ester), to prevent crystal growth and particle aggregation of the composition. The suspension composition can be prepared by suspending 0.01% to 5.0% of the powder composition in a solvent with a pH ranging from 2.5 to 8.5.

The above-described suspension composition can be used to produce a microsuspension composition. The microsuspension composition contains a solvent and the above-described powder composition suspended in the solvent, and has a pH of 2.5 to 8.5. The powder composition suspended in the solvent has an average particle size of less than 500 nm.

In yet another aspect, this invention contemplates a solution containing a solvent and the powder composition described above dissolved in the solvent, wherein the solution has a pH of 8.5 to 12.5 (e.g., 9.0 to 11.0). The solution can contain about 0.01% to 1% of the powder. In some embodiments, the solution is a beverage. It can be prepared by dissolving 0.01% to 1% of the powder in a solvent with a pH of 8.5 to 12.5.

Also included in the present invention is a pharmaceutical composition containing the powder composition described above and a pharmaceutical agent (e.g., a compound for treating a disorder or for modulating a biological process in a human or other mammalian subjects). For example, the pharmaceutical agent can be a cholesterol-lowering agent, an antidiabetic agent, an anticancer agent, an antiviral agent, a COX-1 inhibitor, a COX-2 inhibitor, an hypertension-lowering agent, an antibacterial agent, an anti-inflammatory and gastroprotective agent, an NF-kB modulating agent, a glucose intestine absorption inhibitor, a nitric oxide inhibitor, a PGE-2 inhibitor and a tyrosine kinase inhibitor.

A nutritional supplement is also within the scope of this invention. The supplement includes the above-described powder composition, and optionally, one or more nutrients. The nutrients include, but are not limited to, a vitamin, caffeine, resveratrol, curcumin, catechins, genistein, luteolin, astaxanthin, synepherine, folic acid, rutin, isoquercetin, xanthohumol, humulone, cohumulone, isohumulone, eicosapentaenoic acid (EPA), and docosahexaenoic acid (DHA).

The present invention further contemplates a cosmetic composition containing the powder composition described above. The cosmetic composition can be formulated as, e.g., a gel, cream, liquid, ointment, and powder.

Set forth below is a method of producing an edible powder composition of the present invention. The method includes dissolving a 3-hydroxyflavone (e.g., quercetin) and a water-soluble antioxidant in an aqueous alkali metal hydroxide to produce an alkali metal salt solution. An organic acid, e.g., a weak organic acid, is used to adjust the pH of this solution to a pH of 9.0 to 11.5. A water-soluble anti-deliquescent agent is also added to the alkali metal salt solution. The final solution is then dried, using methods known in the art (e.g., spray drying), to produce the powder composition. In some embodiments, 1:1 to 2:1 w/w of quercetin and the water-soluble antioxidant are dissolved in 1N aqueous sodium hydroxide or potassium hydroxide. In some cases, 5-20% of the water-soluble anti-deliquescent agent is first dissolved in hot water before being added to the alkali metal salt solution.

The details of one or more embodiments of the invention are set forth in the accompanying drawing and the description below. Other features, objects, and advantages of the invention will be apparent from the description and drawing, and from the claims.

### DETAILED DESCRIPTION

Described herein is a flavonol powder composition with improved water solubility and bioavailability. Flavonols with a hydroxyl (OH) moiety at the C-3 position, i.e., 3-hydroxyflavones, can be used to produce the powder composition. Exemplary 3-hydroxyflavones useful for the present invention are listed in Table 1 below.

TABLE 1

| NAME | IUPAC NAME | 5 | 6 | 7 | 8 | 2′ | 3′ | 4′ | 5′ | 6′ |
|---|---|---|---|---|---|---|---|---|---|---|
| 3- | 3-hydroxy-2-phenylchromen-4-one | H | H | H | H | H | H | H | H | H |
| hydroxyflavone | | | | | | | | | | |
| Azaleatin | 2-(3,4-dihydroxyphenyl)-3,7- | OCH₃ | H | OH | H | H | H | OH | OH | H |
| | dihydroxy-5-methoxychromen-4- | | | | | | | | | |
| | one | | | | | | | | | |
| Fisetin | 3,3′,4′,7-tetrahydroxy-2- | H | H | OH | H | H | H | OH | OH | H |
| | phenylchromen-4-one | | | | | | | | | |
| Galangin | 3,5,7-trihydroxy-2-phenylchromen- | OH | H | OH | H | H | H | H | H | H |
| | 4-one | | | | | | | | | |
| Gossypetin | 2-(3,4-dihydroxyphenyl)-3,5,7,8- | OH | H | OH | OH | H | OH | OH | H | H |
| | tetrahydroxychromen-4-one | | | | | | | | | |
| Kaempferide | 3,5,7-trihydroxy-2-(4- | OH | H | OH | H | H | H | OCH₃ | H | H |
| | methoxyphenyl)chromen-4-one | | | | | | | | | |
| Kaempferol | 3,4′,5,7-tetrahydroxy-2- | OH | H | OH | H | H | H | OH | H | H |
| | phenylchromen-4-one | | | | | | | | | |
| Isorhamnetin | 3,5,7-trihydroxy-2-(4-hydroxy-3- | OH | H | OH | H | H | OCH₃ | OH | H | H |
| | methoxyphenyl)chromen-4-one | | | | | | | | | |
| Morin | 2-(2,4-dihydroxyphenyl)-3,5,7- | OH | H | OH | H | OH | H | OH | H | H |
| | trihydroxychromen-4-one | | | | | | | | | |
| Myricetin | 3,3′,4′,5′,5,7-hexahydroxy-2- | OH | H | OH | H | H | OH | OH | OH | H |
| | phenylchromen-4-one | | | | | | | | | |
| Natsudaidain | 2-(3,4-dimethoxyphenyl)-3- | OCH₃ | OCH₃ | OCH₃ | OCH₃ | H | H | OCH₃ | OCH₃ | H |
| | hydroxy-5,6,7,8- | | | | | | | | | |
| | tetramethoxychromen-4-one | | | | | | | | | |
| Pachypodol | 5-hydroxy-2-(4-hydroxy-3- | OH | H | OCH₃ | H | H | H | OH | OCH₃ | H |
| | methoxyphenyl)-3,7- | | | | | | | | | |
| | dimethoxychromen-4-one | | | | | | | | | |
| Quercetin | 3,3′,4′,5,7-pentahydroxy-2- | OH | H | OH | H | H | OH | OH | H | H |
| | phenylchromen-4-one | | | | | | | | | |
| Rhamnazin | 3,5-dihydroxy-2-(4-hydroxy-3- | OH | H | OCH₃ | H | H | OCH₃ | OH | H | H |
| | methoxyphenyl)-7- | | | | | | | | | |
| | methoxychromen-4-one | | | | | | | | | |
| Rhamnetin | 2-(3,4-dihydroxyphenyl)-3,5- | OH | H | OCH₃ | H | H | OH | OH | H | H |
| | dihydroxy-7-methoxychromen-4- | | | | | | | | | |
| | one | | | | | | | | | |

One of the exemplary 3-hydroxyflavones shown in Table 1, quercetin, has the following structure:

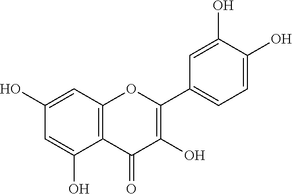

The OH moiety at the C-3 position of a 3-hydroxyflavone has a relatively lower pK value (i.e., 8.0-9.0) as compared to the pK values (i.e., 10.0-11.5) of the OH moieties on other positions. It was unexpectedly discovered that a powder of a 3-hydroxyflavone alkali metal salt with the alkali metal at the C-3 position has high water solubility:

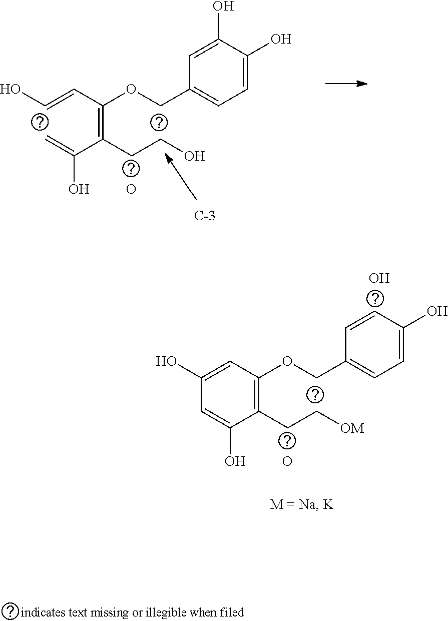

To increase stability of the powder, a water-soluble antioxidant such as an alkali metal salt of vitamin C, can be included in the powder. The powder can further include a water-soluble anti-deliquescent agent, e.g., a gelatin or a polysaccharide. In addition, the powder can contain two or more, e.g., two, three, four or five, alkali metal salts of different 3-hydroxyflavones.

Generally, the powder composition of the present invention can be made by dissolving a 3-hydroxyflavone in an aqueous alkali metal hydroxide (e.g., aqueous sodium hydroxide or potassium hydroxide). The pH of the resulting 3-hydroxyflavone alkali metal salt solution is then adjusted to 9.0-11.5 by adding an organic acid, e.g., citric acid, lactic acid, fumaric acid, acetic acid, tartaric acid, malic acid, and tannin acid. The salt solution can then be dried using conventional methods such as spray drying, lyophilization or evaporation. Before the drying step, an antioxidant, e.g., vitamin C, gallic acid, glutathione, uric acid, lipoic acid, Chlorogenic acid, and ferulic acid, can also be dissolved in the alkaline water together with the 3-hydroxyflavone. Alternatively, the antioxidant is separately dissolved in an aqueous alkali metal hydroxide to produce a solution (pH=9.5 to 11) before being added to the 3-hydroxyflavone alkali metal salt solution. Optionally, a water-soluble anti-deliquescent agent can be added to the salt solution before the drying step. Those of ordinary skill in the art would ready understand that the ingredients used to produce the powder composition should be compatible with human or animal consumption.

The powder composition described above, when added to an aqueous medium, produces either a clear solution or a uniform suspension, depending on the pH of the aqueous medium. The present invention also contemplates such solution or suspension. When the powder of the present invention is added to an aqueous medium having a pH of, e.g., 2.5 to 8.5, a suspension with very fine particles can be obtained. This suspension can be a starting source for preparing microsuspensions, microemulsions and microencapsulations by using various sizing equipments and methods known in the art. See, e.g. U.S. Pat. No. 5,290,654; and Eur J Pharm Biopharm, 69:948-57 (2008); and Arch Pharm Res, 26:426-31 (2003).

A surfactant or a water-soluble polymer can be added to the suspension composition to prevent crystal growth and particle aggregation of the composition.

The powder, when added to an alkaline aqueous medium (e.g., having a pH of 8.5 to 12.5) produces a clear solution. This improved solubility of the powder renders it suitable for adding it to various aqueous media, e.g., sports drink and soda.

The above-described powder composition, suspension composition, microsuspension composition and solution can be used in a variety of applications. For example, they can be formulated as pharmaceutical compositions or nutritional supplements using methods known in the art. The pharmaceutical compositions and supplements can take the form of solutions, suspensions, tablets, pills, capsules, sustained release formulations or powders.

The pharmaceutical compositions, in addition to the compositions and solutions of the present invention, can also contain other pharmaceutical agents, e.g., drugs. Flavonols have been found to play roles in a number of biological activities. In one preferred embodiment, other agents that modulate the same activities or show synergistic effects with flavonols can be included in the pharmaceutical compositions. These agents include, but are not limited to, cholesterol-lowering agents, antidiabetic agents, anticancer agents, antiviral agents, COX-1 inhibitors, COX-2 inhibitors, hypertension-lowering agents, antibacterial agents, anti-inflammatory and gastroprotective agents, NF-kB modulating agents, glucose intestine absorption inhibitors, nitric oxide inhibitors, PGE2 inhibitors, and tyrosine kinase inhibitors.

The nutritional supplements can contain the 3-hydroxyflavone compositions and solutions described herein and optionally one or more other nutrients described above.

The compositions and solutions of the present invention can be added to various edible compositions, such as beverages, soft chews, chewing gums, candies, and foods. They can also be formulated as creams, lotions, gels, ointments and liquids for oral hygiene, skin care, cosmetics, and other topical applications. The compositions can also be formulated for administration to skin or mucosal tissue as, e.g., nasal sprays, bronchial inhalers (liquid or powder), and vaginal or rectal suppositories. In the case of allergy treatment, administration can be accomplished by use of an inhaler or atomizer. Similarly, compositions suitable for administration to the eye or ear (such as ophthalmic or optic drops) can be formulated using methods known in the art.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are incorporated herein by reference in their entirety.

### Example 1

### Preparation of a Water-Soluble Powder of Quercetin

15 g of quercetin (98%; purchased from Aldrich) and 15 g of vitamin C (99%; purchased from Aldrich) were dissolved in 1N NaOH (213 mL). 2% acetic acid (290 mL) was added to the solution to adjust the pH to 10.8 under stirring. 2.5 g of cluster dextrin (Ezaki Glico Co, Japan) and 5 g of acid-treated porcine gelatin were then added to the solution. The final 520 mL aqueous solution (pH=10.5) was subjected to spray drying, resulting in 40 g of a pale orange powder.

### Example 2

### Preparation of a Water-Soluble Powder of Quercetin

3 g of quercetin (98%; purchased from Aldrich) and 3 g of vitamin C (99%; purchased from Aldrich) were dissolved in 1N KOH (45 mL). 10% tannic acid (30 mL) was then added to adjust the pH of the solution to pH 10.8. 3.8% aqueous acid-treated porcine gelatin (40 mL) was added to the solution. The final 120 mL aqueous solution (pH=10.0) was spray dried, resulting in 12 g of a pale orange powder.

### Example 3

### Preparation of a Water-Soluble Powder of Quercetin

15 g of quercetin (98%; purchased from Aldrich) and 15 g of vitamin C (99%; purchased from Aldrich) were dissolved in 1N NaOH (200 mL). 1% citric acid (30 mL) and then 2% acetic acid (70 mL) were added to the solution to adjust the pH to 10.8 under stirring. 8.3 g of maltodextrin was then added to the solution. The final 400 mL aqueous solution (pH=10.5) was subjected to spray drying, producing 40 g of a pale orange powder.

### Example 4

### Preparation of a Water-Soluble Powder of Quercetin

3 g of quercetin (98%; purchased from Aldrich) and 3 g of ascorbic acid (i.e., vitamin C; 99%; purchased from Aldrich) were dissolved in 1N NaOH (45 mL) to produce a salt solution. 2% acetic acid (40 mL) was added to this salt solution to adjust the pH of the solution to 10.8. Next, 3.8% aqueous acid-treated porcine gelatin (40 mL) was added to the solution. The resulting 120 mL aqueous solution (pH=10.5) was then subjected to spray drying, producing 7.5 g of a pale orange powder.

### Example 5

### Preparation of a Suspension of Quercetin

1 g of the quercetin alkali metal salt powder prepared in Example 1 was added to 50 mL water with a pH of 2.6, 5.5, 7.2 or 8.5 to produce samples B, C, D and E, respectively. A control sample, sample A, was prepared by adding 1 g of a quercetin powder (purchased from Aldrich) to 50 mL of water at pH 8.5. Samples D and E were clear solutions. Sample B (pH 3.8) and sample C (pH 6.5) were uniform suspensions without precipitation. The particles in samples B and C looked very fine, as compared to sample A, which contained non-uniform lumps of the powder. Thus, the quercetin alkali metal salt powder prepared in Example 1 is surprisingly more water-soluble than quercetin.

### Example 6

### Stability Study of Quercetin Powders Under a Force Condition

The following quercetin powders were prepared using the methods described in Examples 1-4:

Sample 1: Quercetin Na salt (70%)+maltodextrin (15%)
Sample 2: Quercetin Na salt (28%)+Vitamin C (28%)+acid treated gelatin (15%)
Sample 3: Quercetin Na salt (28%)+Vitamin C (28%)+maltodextrin (10%)
Sample 4: Quercetin Na salt (28%)+vitamin C (29%)+Cluster dextrin (15%)

Each sample (5 g) was placed in a clear glass jar (volume=100 mL) with the lid on. The glass jars were placed in a thermal chamber (at 75° C.) and, at various time points, the amount of quercetin in each sample was determined by HPLC. The data are shown in Table 2 below. The combination of quercetin with vitamin C and acid treated gelatin was found to be the most stable formulation under the force condition.

TABLE 2

| | Extrapolated time | Sample | Sample | Sample | Sample |
|---|---|---|---|---|---|
| | (25° C.) | 1 | 2 | 3 | 4 |
| Day 0 | | 100% | 100% | 100% | 100% |
| Day 7 | 8 month | 0 | 98% | 56% | 85% |
| Day 12 | 1 year | 0 | 98% | 54% | 82% |
| Day 24 | 2 year | 0 | 96% | 52% | 82% |

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. An edible powder composition comprising:
an alkali metal salt of a 3-hydroxyflavone;
an alkali metal salt of an organic acid; and
a water-soluble antioxidant.

2. The powder of claim 1, further comprising a water-soluble anti-deliquescent agent.

3. The powder composition of claim 2, when dissolved in water at 0.2%/w/v, results in a solution that has a pH of 8.5 to 11.5.

4. The powder composition of claim 2, wherein the alkali metal salt of a 3-hydroxyflavone is 10% to 40%; the anti-deliquescent agent is 2% to 20%; and the antioxidant is 10% to 40%.

5. The powder composition of claim 2, wherein the 3-hydroxyflavone is selected from the group consisting of quercetin, azaleatin, fisetin, galangin, gossypetin, kaempferide, kaempferol, isorhamnetin, mortin, myricetin, natsudaidain, pachypodol, rhamnazin, and rhamnetin.

6. The powder composition of claim 5, wherein the 3-hydroxyflavone is quercetin.

7. The powder composition of claim 6, further comprising an alkali metal salt of a different 3-hydroxyflavone.

8. The powder composition of claim 7, wherein the different 3-hydroxyflavone is selected from the group consisting of azaleatin, fisetin, galangin, gossypetin, kaempferide, kaempferol, isorhamnetin, mortin, myricetin, natsudaidain, pachypodol, rhamnazin, and rhamnetin.

9. The powder composition of claim 1, wherein the alkali metal in each of the alkali metal salt of a 3-hydroxyflavone and the alkali metal salt of an organic acid is sodium or potassium.

10. The powder composition of claim 2, wherein the anti-deliquescent agent is a gelatin or a polysaccharide.

11. The powder composition of claim 10, wherein the anti-deliquescent agent is acid treated porcine-derived gelatin, maltodextrin, or cluster dextrin.

12. The powder composition of claim 1, wherein the organic acid is selected from the group consisting of citric acid, lactic acid, fumaric acid, acetic acid, tartaric acid, malic acid, and tannin acid.

13. The powder composition of claim 1, wherein the antioxidant is selected from the group consisting of vitamin C, gallic acid, glutathione, uric acid, lipoic acid, chlorogenic acid, and ferulic acid.

14. The powder composition of claim 2, wherein
the alkali metal salt of a 3-hydroxyflavone is quercetin potassium salt or quercetin sodium salt;
the alkali metal salt of an organic acid is acetic acid potassium salt or acetic acid sodium salt; and
the antioxidant is vitamin C potassium salt or vitamin C sodium salt; and
the anti-deliquescent agent is acid treated porcine gelatin.

15. The powder composition of claim 14, wherein the alkali metal salt of a 3-hydroxyflavone is 15% to 35%; the anti-deliquescent agent is 5% to 20%; and the antioxidant is 15% to 35%.

16. A suspension composition comprising a solvent and the powder composition of claim 1 suspended in the solvent, the suspension composition having a pH of 2.5 to 8.5.

17. The suspension composition of claim 16, comprising 0.01% to 5% of the powder of claim 1.

18. The suspension composition of claim 16, further comprising a surfactant or a water-soluble polymer to prevent crystal growth and particle aggregation of the composition.

19. A microsuspension composition comprising a solvent and the powder composition of claim 1 suspended in the solvent, the microsuspension composition having a pH of 2.5 to 8.5, wherein the powder composition suspended in the solvent has an average particle size of less than 500 nm.

20. The microsuspension composition of claim 19, comprising 0.01% to 5% of the powder of claim 1.

21. The microsuspension of claim 19, further comprising a surfactant or a water-soluble polymer to prevent crystal growth and particle aggregation of the composition.

22. A solution comprising a solvent and the powder composition of claim 1 dissolved in the solvent, wherein the solution has a pH of 8.5 to 12.5.

23. The solution of claim 22, comprising 0.01% to 1% of the powder of claim 1.

24. A pharmaceutical composition comprising the powder composition of claim 6 and a pharmaceutical agent

25. The pharmaceutical composition of claim 24, wherein the pharmaceutical agent is selected from the group consisting of a cholesterol-lowering agent, an antidiabetic agent, an anticancer agent, an antiviral agent, a COX-1 inhibitor, a COX-2 inhibitor, an hypertension-lowering agent, an antibacterial agent, an anti-inflammatory and gastroprotective agent, an NF-kB modulating agent, a glucose intestine absorption inhibitor, a nitric oxide inhibitor, a PGE-2 inhibitor and a tyrosine kinase inhibitor.

26. A nutritional supplement comprising the powder composition of claim 6 and a nutrient.

27. The nutritional supplement of claim 26, wherein the nutrient is selected from the group consisting of a vitamin, resveratrol, curcumin, catechins, genistein, luteolin, astaxanthin, synepherine, steviosides, folic acid, rutin, isoquercetin, caffeine, xanthohumol, humulone, cohumulone, isohumulone, eicosapentaenoic acid (EPA), and docosahexaenoic acid (DHA).

28. A cosmetic composition comprising the powder of claim 6.

29. A method of producing an edible powder composition, the method comprising:
producing a solution including an aqueous alkali metal hydroxide, quercetin, a water-soluble antioxidant, and optionally, a water-soluble anti-deliquescent agent, wherein the solution has a pH of 9.0 to 11.5; and
drying the solution to produce the powder composition.

30. The method of claim 29, wherein the producing step is performed by dissolving 1:1 to 2:1 w/w of quercetin and the water-soluble antioxidant in 1 N aqueous sodium hydroxide or potassium hydroxide, the pH of the solution being adjusted by adding an organic acid.

31. The method of claim 29, wherein 5-20% of the water-soluble anti-deliquescent agent is first dissolved in hot water before being added to the solution.

32. The method of claim 29, wherein the drying step is spray drying.
